(19) United States
(12) Patent Application Publication
(10) Pub. No.: US 2022/0241230 A1
(43) Pub. Date: Aug. 4, 2022

(54) DICLOFENAC SACHET COMPOSITION

(71) Applicant: Patrin Pharma, Inc., Skokie, IL (US)

(72) Inventors: Jay Sanjay Trivedi, Skokie, IL (US); Mandar Madhukar Kodgule, Navi Mumbai (IN); Sharad Shridhar Darandale, Navi Mumbai (IN); Rashmita Duryodhan Ella, Mumbai (IN)

(73) Assignee: Patrin Pharma, Inc., Skokie, IL (US)

(21) Appl. No.: 17/591,333

(22) Filed: Feb. 2, 2022

Related U.S. Application Data
(60) Provisional application No. 63/145,421, filed on Feb. 3, 2021.

(51) Int. Cl. A61K 31/196; A61K 9/00; A61K 9/16

(52) U.S. Cl. CPC A61K 31/196; A61K 9/0053; A61K 9/009; A61K 9/1641; A61K 9/1623

(57) ABSTRACT

A pharmaceutical composition for oral administration comprising a free-flowing granular powder comprising diclofenac or a pharmaceutically acceptable salt thereof, a pharmaceutically acceptable substantially non-hygroscopic water soluble diluent, an optional binder, and one or more optional pharmaceutically acceptable excipients, wherein the composition is devoid of any alkalizer, buffer or base. The pharmaceutical composition is divided into unit doses containing a therapeutically effective amount of diclofenac, and may be incorporated into sachets, tablets or capsules. and not less than 85% of the free-flowing granulate powder dissolves after 3 minutes in simulated intestinal fluid at pH 6.8. The pharmaceutical composition is rapidly bioavailable when orally administered to a mammal, e.g., human.

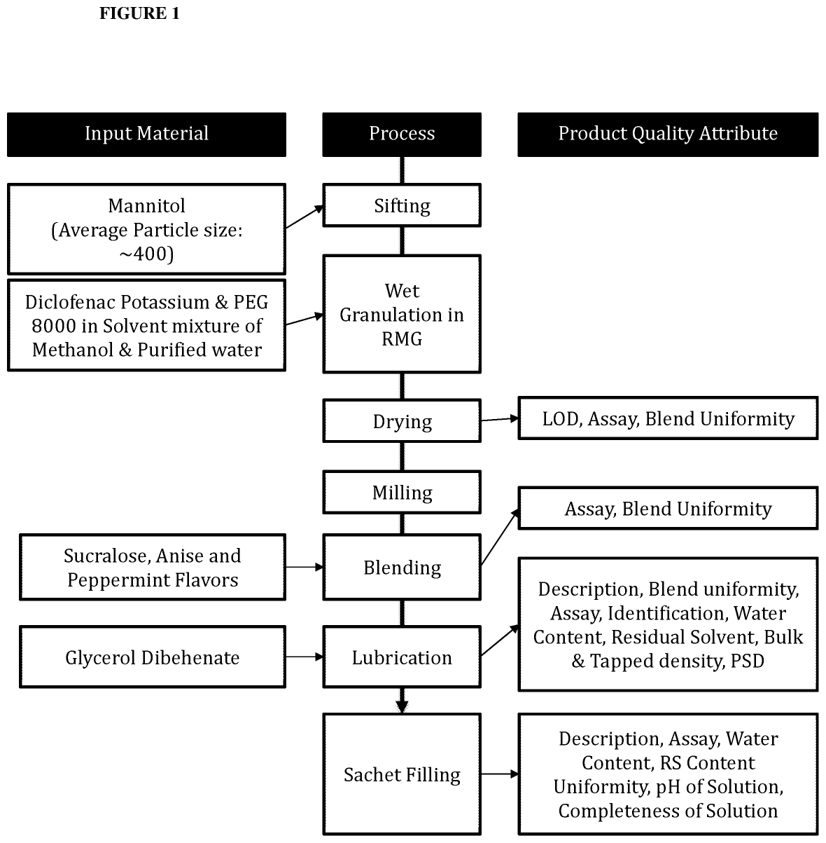

### FIELD OF THE INVENTION

The present invention provides a pharmaceutical composition for treating migraine and other acute pain episodes in a mammal (e.g. human) which includes diclofenac or pharmaceutically acceptable salt thereof in poser sachets. The invention further concerns methods and formulations for treating symptoms that often accompany migraine and acute pain such as rebound headache, photophobia, phonophobia, nausea and vomiting.

### BACKGROUND OF THE INVENTION

Diclofenac is a non-steroidal anti-inflammatory drug (“NSAID”) known chemically as [(2,6-dichloro-anilino)-2-phenyl]-2-acetic acid.

Diclofenac is widely used for treating various types of pain, including both chronic and acute painful episodes. The drug is administered for the treatment of musculoskeletal and joint disorders such as rheumatoid arthritis, osteoarthritis, and ankylosing spondylitis; periarticular disorders such as bursitis and tendonitis; soft tissue disorders such as sprains and strains, and other painful conditions such as renal colic, acute gout, dysmenorrhoea, and following some surgical procedures. Reynolds, Diclofenac In The Extra Pharmacopoeia, London: The Pharmaceutical Press; p. 31-33 (Martindale (2000). Diclofenac has also been studied for the treatment of headache pain from migraine attacks, using various doses and dosage forms, including 75 mg intramuscular injections (Del Bene et al., Intramuscular treatment of migraine attacks using diclofenac sodium: a cross-over trial, J. Int. Med. Res., pp. 1544-8 (1987), 100 mg suppositories (Del Bene et al., Migraine attack treatment with diclofenac sodium. Cephalalgia, 5:144-5, (1985), and 50 mg enteric coated tablets. (Massiou et al., Effectiveness of oral diclofenac in the acute treatment of common migraine attacks: a double blind study versus placebo, Cephalalgia, 1:59-63 (1991).

Migraine attacks manifest a diverse array of symptoms that must be resolved in order for a treatment to be deemed truly effective against migraine (instead of just treating the symptoms). In particular, the treatment must be effective against the pain, photophobia, phonophobia and nausea that are caused by migraine, and it must be effective within the first two hours of treatment, in order to be considered a true treatment for migraine.

In 1993, investigators studied 100 mg and 50 mg diclofenac tablets, in comparison to placebo, and determined that both strengths were effective against migraine pain within two hours of treatment, but that only the 100 mg strength was effective against phonophobia and photophobia within two hours. (Dahlöf et al., Diclofenac-K (50 and 100 mg.) and placebo in the acute treatment of migraine, Cephalalgia, 13:117-123, (1993).

In 1999, a separate group of investigators tested 50 mg and 100 mg sugar coated tablets of diclofenac potassium to treat migraine and once again confirmed the ability of both doses to relieve migraine pain within two hours of treatment. (The Diclofenac-K/Sumatriptan Migraine Study Group, Acute treatment of migraine attacks: efficacy and safety of a nonsteroidal anti-inflammatory drug, diclofenac potassium, in comparison to oral sumatriptan and placebo, Cephalalgia, 19:232-40 (1999). The investigators concluded that neither dose was effective against photophobia two hours after treatment, that both doses were effective against photophobia eight hours after treatment, that only the 100 mg dose was effective against phonophobia two hours after treatment, and that the 50 mg dose was effective against photophobia eight hours after treatment. These investigators also studied the effectiveness of 100 mg and 50 mg diclofenac-K immediate release tablets at preventing recurrence of headaches within 48 hours of treatment. The investigators concluded that patients treated with the 50 mg and the 100 mg diclofenac-K tablets actually had a higher incidence of headache recurrence than patients treated with placebo (i.e., that the diclofenac-K performed worse than placebo), although the statistical significance of these findings is not reported.

This latter finding is consistent with other literature which recommends the use of a “long acting NSAID” to reduce the frequency of rebound headaches. For example, U.S. Pat. No. 6,586,458 (Plachetka) recommends that triptan therapy be augmented with a “long acting NSAID” to provide “a substantial reduction in the frequency [of] relapse of headaches.” Diclofenac potassium is not considered a long acting NSAID because it displays an average Cₘₐₓ within only about one hour and a terminal half-life of only about 1.9 hours when administered in commercially available sugar coated tablets.

Diclofenac is generally taken orally in the form of normal tablets or tablets covered with coatings resistant to gastric juices, or rectally, or by injection, or topically. Recently, however, in WO 97/44023 (Reiner et al.) it is proposed to administer diclofenac in a number of less conventional dosage forms including as a powder sachet for oral administration after dissolving in water for quicker onset of analgesic relief.

One of the primary obstacles in the manufacture of powder sachets is the distribution of the drug in the powder, and the uniformity of content in the finished product. These hurdles are magnified in the production of diclofenac sachets due to the poor aftertaste of diclofenac and the need to incorporate additional ingredients to compensate for this poor taste.

To ensure an adequately homogenous distribution of drug product in the bulk powder, the Reiner et al. patent filing describes a wet granulation process for manufacturing the powder sachets. In the first step of the process, a wet granulate is prepared from diclofenac potassium, potassium bicarbonate, saccharin, aspartame and mannitol, using 95% ethanol as the wetting agent. The granulate is then mixed with over one gram of sugar (saccharose) and various flavoring agents to improve the taste of the composition.

The method described in the Reiner et al. patent filing produces an excellent pharmaceutical dosage form, but suffers from a number of disadvantages including the size of the sachet (2 g) which makes the sachet more difficult to dissolve, and the presence of sugar in the formulation, which should be avoided in the diabetic population. In addition, the process requires precise controls on the granulometric process to assure uniform distribution of drug in the granulate and consistent amounts of drug in the finished product.

A wet granulated formulation of diclofenac potassium has been developed to provide an increased rate of absorption, and its pharmacokinetic properties tested against commercially available diclofenac potassium tablets. (Reiner et al., Arzniem,—Forsch,/Drug Res., 51:885-890 (2001). According to the authors, the granular formulation showed a higher Cₘₐₓ than the diclofenac potassium tablets, a shorter tₘₐₓ (i.e. time to Cₘₐₓ) and a similar AUC when compared to the tablet form.

The method described by Renier et al. in U.S. Pat. Nos. 7,759,394; 8,097,651; 8,927,604 and 9,827,197, produced by the large proportion of mannitol, imparts rapid bioavailability to the formulation, while the control of particle size assures uniform distribution of diclofenac in the material used to fill the sachets and consistent amounts of drug in each sachet without the use of sugar or large amounts of diluent as taught in the prior art. The method and powders produced by the method are characterized by, among other variables, (1) the ratio of the diluent to the diclofenac in the powder, (2) a combination of particle sizes of the diluent in the final composition, and (3) the sequence of mixing the diclofenac and the varying particle sizes of diluent. Buffering agents are not critical to the formulation described therein, but are preferably used to provide a rapid rate of onset for the final pharmaceutical product. In a preferred embodiment for powder sachets, the buffering agent controls the pH of the formulation when dissolved in water, and preferably yields a pH greater than about 6.8, 7.0, 7.2, or 7.4, and less than about 7.8, 7.7 or 7.6, when mixed with 50 ml or 100 or 200 ml. of water at 25 degrees Celsius.

Diclofenac potassium for oral solution is commercially available in the U.S. under the tradename Cambia® as a single 50 mg dose packet. The contents are mixed with 1-2 ounces or 2-4 tablespoons (30 to 60 ml) of water prior to administration.

What is needed is an alternative method for producing and pharmaceutical formulation of powder diclofenac sachets with uniform particle size of granules, i.e., having a coarse particle size of more than about 300 microns and to achieve a pH near to about pH 7 without using a buffering agent and other fast acting dosage forms of diclofenac.

### OBJECTS AND SUMMARY OF THE INVENTION

It is an object of the invention to provide improved methods and formulations for treating or preventing headache pain from migraine attacks.

It is another object of the present invention to provide a method for treating or preventing the pain, photophobia, phonophobia and nausea that are caused by migraine, which is preferably effective within the first two hours after administration to a human in need of treatment therefor.

It is another object of the present invention to overcome or ameliorate at least one of the disadvantages of the prior art, or to provide a useful alternative.

It is a further object of the present invention to provide a rapidly bioavailable formulation of diclofenac which does not include alkalizer, buffer or base.

It is a further object of the present invention to provide a rapidly bioavailable formulation of diclofenac which has product uniformity.

In accordance with the above objects and others, the invention is directed in part to a pharmaceutical composition for oral administration comprising a free-flowing granular powder comprising diclofenac or a pharmaceutically acceptable salt thereof and a pharmaceutically acceptable substantially non-hygroscopic, water soluble diluent, and one or more optional pharmaceutically acceptable excipients, not less than 85% of the free-flowing granulate powder dissolves after 3 minutes in simulated intestinal fluid at pH 6.8. In certain embodiments, the pharmaceutical composition is devoid of any alkalizer, buffer or base.

The invention is further directed to a pharmaceutical composition for oral administration comprising a free-flowing granular powder comprising diclofenac or a pharmaceutically acceptable salt thereof, a pharmaceutically acceptable diluent (which is preferably non-hygroscopic and freely water soluble), and a pharmaceutically acceptable binder. In certain preferred embodiments, the free-flowing granular powder is contained in a sachet containing a therapeutically effective dose of diclofenac or a pharmaceutically acceptable salt thereof. In certain preferred embodiments, a unit dose of the granular powder is greater than 75% or 85% soluble or is completely solubilized in 50 ml of water at 25 degrees Celsius, e.g., in less than about 3 minutes. In certain preferred embodiments, the diclofenac is wet-granulated, e.g., with (i) the binder, (ii) the diluent, or (iii) both the binder and the diluent to obtain agglomerated particles containing the diclofenac. In certain preferred embodiments, the pharmaceutical composition does not include any alkalizer, buffer or base. In further preferred embodiments, the diluent is a non-hygroscopic and is freely soluble in water. In certain preferred embodiments, the diluent has an average particle diameter above about 350 microns, and more preferably has an average particle diameter from about 400 to about 750 microns, and in certain preferred embodiments from about 650 to about 750 microns, and in certain other preferred embodiments from about 400 to about 600 microns.

The invention is further directed, in part, to a pharmaceutical composition for oral administration comprising a free-flowing granular powder comprising from about 25 to about 75 mg of diclofenac or a pharmaceutically acceptable salt thereof, from about 300 to about 800 mg of mannitol having average particle diameter preferably above about 350 microns along with other pharmaceutically acceptable excipient. In certain embodiments, the composition is devoid of any alkalizer, buffer or base. In certain preferred embodiments, the mannitol has an average particle diameter from about the mannitol has an average particle diameter from about 400 to about 750 microns. In certain preferred embodiments, the pharmaceutical composition is in the form of a sachet. In certain preferred embodiments, the pharmaceutically acceptable excipient comprises a binder. In certain preferred embodiments, the binder is a polyethylene glycol (PEG) such as PEG 8000. In certain preferred embodiments, the pharmaceutically acceptable excipient further comprises a diluent. In certain preferred embodiments, the pharmaceutical composition comprises agglomerated particles of diclofenac, mannitol and one or more pharmaceutically acceptable excipients which is not an alkalizer, buffer or base, e.g., a binder. In certain preferred embodiments, the water content of the pharmaceutical composition is preferably less than about 2%. In certain preferred embodiments, the pharmaceutically acceptable excipient further comprises a sweetener (preferably a non-sugar sweetener) and/or a flavoring agent. In certain preferred embodiments, the pharmaceutically acceptable excipient further comprises a lubricant. In certain preferred embodiments, the amount of diclofenac in each sachet is from about 25 mg to about 75 mg, based on diclofenac potassium. In certain preferred embodiments, the solvent is an alcoholic (e.g., methanol) or hydroalcoholic solvent (e.g., a mixture of methanol and (purified) water). In certain preferred embodiments, the average particle size of the free-flowing granular powder is from about 350 microns to about 900 microns, and most preferably from about 350 microns to about 750 microns. In certain preferred embodiments, the free-flowing granular powder obtained by the method includes from about 5% to about 10% diclofenac (based on the potassium salt), from about 60% to about 95% diluent (e.g., mannitol), and from about 0.1% to about 3% binder (e.g., polyethylene glycol). These percentages are particularly useful for a pharmaceutical composition (unit dose) which as a total weight from about 700 mg to about 1 g. One skilled in the art will understand that if significantly lesser or higher quantities of total weight, the percentage of each ingredient may change significantly. All percentages are w/w unless otherwise indicated. In certain preferred embodiments, a unit dose of the granular powder is greater than 75% or 85% soluble or is completely solubilized in 50 ml of water at 25 degrees Celsius, e.g., in less than about 3 minutes.

In another embodiment the product is characterized by the total amount of powder used to fill a sachet, which is preferably greater than 500, 600, 700 or 800 mg, and/or less than 1800, 1600, 1400, 1200, or 1000 mg, based on a 50 mg diclofenac sachet. In certain embodiments, a preferred amount of powder contained in each sachet is about 900 mg and the amount preferably does not vary outside the range from about 865 mg to about 925 mg per sachet range (per package). In one embodiment a unit dose of the sachet is greater than 75% or 85% soluble or is completely soluble in 50 ml of water at 25 degrees Celsius. In another embodiment the unit dose is greater than 75% or 85% solubilized or is completely solubilized in 50 ml of water with stirring at 25 degrees Celsius in less than 3 minutes. In a preferred embodiment, the dissolution profile of the dosage forms of the present invention is not less than 85% after 3 minutes in simulated intestinal fluid (i.e. water) at pH 6.8.

The invention is further directed to a method of preparing powder diclofenac sachets, comprising adding a pharmaceutically acceptable binder to an alcoholic or hydroalcoholic solvent; adding diclofenac or a pharmaceutically acceptable salt thereof to the mixture to obtain a drug-binder dispersion; granulating a diluent with the drug-binder dispersion; drying the resultant mixture to obtain a free-flowing granular powder. In further embodiments, the method further comprises filling the blend into suitably sized sachets containing a therapeutic dose of the diclofenac. In further embodiments, the method further comprises adding a pharmaceutically acceptable lubricant to the free-flowing granular powder prior to filling the blend into suitably sized sachets containing a therapeutic dose of the diclofenac. In certain preferred embodiments, one or more pharmaceutically acceptable excipients are added extragranularly to the free-flowing granular powder. In certain embodiments, the extragranular materials are selected from one or more flavors (e.g., sucralose, peppermint flavor, and/or star anise flavor) and/or a lubricant (e.g., glyceryl dibehenate). In certain preferred embodiments, the amount of diclofenac in each sachet is from about 12.5 mg to about 100 mg, more preferably from about 25 mg to about 75 mg, based on diclofenac potassium. In certain preferred embodiments, the solvent is a mixture of methanol and purified water. In certain preferred embodiments, the average particle size of the free-flowing granular powder is from about 350 microns to about 900 microns, and most preferably about 350 microns to about 750 microns. In certain preferred embodiments, the free-flowing granular powder obtained by the method includes from about 5% to about 10% diclofenac (based on the potassium salt), from about 60% to about 95% diluent (e.g., mannitol), and from about 0.1% to about 3% lubricant (e.g., glycerol dibehenate). All percentages are w/w unless otherwise indicated.

Additional advantages of the invention will be set forth, in part, in the description which follows, and, in part, will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### Definitions

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

“Effective amount” or “therapeutically effective amount” are used interchangeably herein, and refer to an amount of a compound, formulation, material, or composition, as described herein effective to achieve a particular biological result. Such results may include, but are not limited to, the treatment of a disease or condition as determined by any means suitable in the art.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to oral administration.

“Pharmaceutically acceptable” refers to those properties and/or substances that are acceptable to the patient from a pharmacological/toxicological point of view and to the manufacturing pharmaceutical chemist from a physical/chemical point of view regarding composition, formulation, stability, patient acceptance and bioavailability.

The term “treat” or “treating”, as used herein, includes, but is not limited to, alleviation or amelioration of one or more symptoms or conditions; diminishment of the extent of disease, disorder, or condition; stabilized (i.e., not worsening) state of the disease, disorder, or condition; preventing spread of the disease, disorder, or condition (e.g., delay or slowing the progress of the disease, disorder, or condition; amelioration or palliation of the disease, disorder, or condition; and remission (whether partial or total), whether detectable or undetectable.

As used herein, “preventing” includes preventing the initiation of acute pain, e.g., pain from migraine, and/or reducing the severity or intensity of migraine and acute pain and effects thereof such as rebound headache, photophobia, phonophobia, nausea and vomiting.

As used herein, “alleviate” is used interchangeably with the term “treat”. Treating a disease, disorder or condition may or may not include complete eradication or elimination of the symptom.

The term “therapeutic” as used herein means a treatment and/or prophylaxis. Throughout this disclosure, various aspects of the invention can be presented in a range format.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, and 6. This applies regardless of the breadth of the range.

### BRIEF DESCRIPTION OF THE DRAWING

The accompanying drawing, which is incorporated in and constitutes a part of this specification, illustrates several embodiments of the invention and together with the description, serves to explain the principles of the invention.

The FIGURE is a flow diagram illustrating wet granulation method and sequence of mixing employed in making 900 mg powder sachets of the invention that contain 50 mg of diclofenac potassium.

### DETAILED DESCRIPTION OF THE INVENTION

The present invention is directed in part to the surprising discovery that rapidly bioavailable pharmaceutical composition for treating migraine and other acute pain episodes using diclofenac or pharmaceutically acceptable salt thereof, using a simple wet granulation process and mannitol having average particle diameter above 350 microns. In addition, the present inventors surprisingly found that wet granulation is the choice of method for making the rapidly bioavailable formulations of diclofenac. The invention further concerns methods and formulations for treating symptoms that often accompany migraine and acute pain such as rebound headache, photophobia, phonophobia, nausea and vomiting.

As previously mentioned, in certain embodiments, ingredients of the invention (including or not including the diclofenac or salt) are wet granulated. The wet granulation preferably results in a product which has acceptable content uniformity. The individual steps in the wet granulation process include milling and sieving of the ingredients, dry powder mixing, wet massing, granulation, drying, and final grinding. In various embodiments, the active agent(s) of the present invention composition is added to the other excipients of the pharmaceutical formulation during the wet granulation process. Alternatively, the active agent may be added after the other ingredients (e.g., binder and diluent) have been wet granulated. Alternatively, the ingredients may be subjected to dry granulation.

In certain preferred embodiments, the free-flowing granular powder obtained by the method includes from about 5% to about 10% diclofenac (based on the potassium salt), from about 60% to about 95% diluent (e.g., mannitol), and from about 0.1% to about 3% binder (e.g., polyethylene glycol). These percentages are particularly useful for a pharmaceutical composition (unit dose) which as a total weight from about 700 mg to about 1 g, e.g., when the pharmaceutical composition is contained in a sachet. One skilled in the art will understand that if significantly lesser or higher quantities of total weight are included, the percentage of each ingredient may change significantly. All percentages are w/w unless otherwise indicated. In certain preferred embodiments, a unit dose of the granular powder is greater than 75% or 85% soluble or is completely solubilized in 50 ml of water at 25 degrees Celsius, e.g., in less than about 3 minutes.

The pharmaceutical composition of the present invention is preferably a free-flowing powder granulate having an average particle size from about 350 microns to about 900 microns, and more preferably from about 350 microns to about 750 microns. In certain preferred embodiments, the free flowing powder is divided into individual dosage forms in the form of, e.g., sachets, containing a therapeutically effective amount of the diclofenac. In certain embodiments, the therapeutic dose of diclofenac may be administered to a mammal, e.g., a human suffering from a migraine, in multiple sachets.

### Diclofenac

The diclofenac used in the present invention can be defined by various parameters. In one embodiment, the raw material will be a powder that exhibits no more than 0.5 wt. % loss on drying. In another embodiment D (90) of the diclofenac particles are not more than 150 micrometers in diameter, D (50) of the diclofenac particles are not less than 10 micrometers and not more than 60 micrometers in diameter, D (10) of the diclofenac particles are not more than 10 microns in diameter. Analyses may be performed using the Malvern Wet Method.

The diclofenac can be present in acid or salt form although, owing to its poor solubility in water, diclofenac is normally used in salt form. The salts of diclofenac customarily used are those of sodium, potassium or other alkali and alkaline earth metals, together with salts of organic nature, such as the salts of basic amino acids, such as lysine, arginine and ornithine, or other pharmacologically acceptable organic bases which have the ability to render the resulting salt soluble in water. Diclofenac potassium is preferably used in this invention due to its fast onset of action.

In a preferred embodiment, 50 mg of diclofenac or its salt is used in the final dosage form, although other amounts could be used including 12.5, 25, 37.5, 50, 75 or 100 mg of diclofenac, or a range having as endpoints any of the foregoing amounts. The amount of diclofenac preferably does not vary by more than about 95-105% from dose to dose.

### Diluents for Powder Sachets

Diluents or “filler excipients” are preferably added to increase the bulk of the resulting dosage units, and to improve blending characteristics. Freely soluble diluents are particularly preferred because they improve the solubility of the final product. The diluent preferably has a solubility in water at 25 degrees Celsius of greater than about 10, 15 or 20 g/100 ml of water. A particularly preferred diluent is mannitol, which is substantially non-hygroscopic, and which has freely soluble in water. In certain preferred embodiments, the mannitol has an average particle diameter from about 400 to about 750 microns. In certain preferred embodiments, the pharmaceutical composition is in the form of a sachet.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like. Mannitol as described herein is especially preferred.

In the wet granulation method, the diclofenac is granulated along with (at least a portion of) the coarse diluent powder and binding agent before any further processing. This is accomplished so that the product does not have issues of content uniformity and filling.

In certain embodiments, the mannitol used in the pharmaceutical compositions of the invention is a quick dissolving product such as Mannogem® mannitol, commercially available from SPI Pharma. Table 1 below provides a particle size distribution by Sieve Shaker method for a pharmaceutically acceptable diluent, e.g., mannitol (Mannogem® Granular):

TABLE 1

| | Microns | Mannogem ® |
|---|---|---|
| Sieve no(#) | (μm) | Granular |
| 20 | 850 | 0.12 |
| 30 | 600 | 45.51 |
| 40 | 425 | 69.47 |
| 60 | 250 | 89.84 |
| 80 | 180 | 98.83 |
| 100 | 150 | 99.76 |
| 140 | 106 | 99.92 |
| collector | <75 | 100.00 |

### Binder

Binders impart cohesiveness to solid oral dosage form formulations: for powder-filled capsule formulation, they aid in plug formation that can be filled into soft or hard shell capsules and in tablet formulation, binders ensure that the tablet remains intact after compression and help assure blend uniformity prior to a compression or fill step. Materials suitable for use as binders in the pharmaceutical compositions described herein include, but are not limited to, carboxymethylcellulose, methylcellulose (e.g., Methocel®), hydroxypropylmethylcellulose (e.g. Hypromellose USP Pharmacoat-603, hydroxypropylmethylcellulose acetate stearate (Aqoate HS-LF and HS), hydroxyethylcellulose, hydroxypropylcellulose (e.g., Klucel®), ethylcellulose (e.g., Ethocel®), and microcrystalline cellulose (e.g., Avicel®), microcrystalline dextrose, amylose, magnesium aluminum silicate, polysaccharide acids, bentonites, gelatin, polyvinylpyrrolidone/vinyl acetate copolymer, crosspovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinylpyrrolidone (e.g., Povidone® CL, Kollidon® CL, Polyplasdone® XL-10, and Povidone® K-12), larch arabogalactan, Veegum®, polyethylene glycol, waxes, sodium alginate, and the like. A particularly preferred binder is polyethylene glycol (PEG).

It has been found that the use of the binder allows the diclofenac to adhere to the mannitol particles (e.g., during the wet granulation phase of manufacture). Without binder, it is impossible to get the drug adhering to the mannitol particles which achieves content uniformity.

### Lubricant

In the method of manufacturing the product, the lubricant is preferably mixed with the granular material (agglomerated mixture of diclofenac and diluent).

While the use of lubricants is not strictly necessary, in certain preferred embodiments, a pharmaceutically acceptable lubricant is added to the powder to prevent the powder from sticking to the metering machine in the final stage of filling the sachets. Suitable lubricants include magnesium stearate, stearic acid, hydrogenated castor oil, talc, or mixtures thereof, but a preferred lubricant is glycerol dibehenate. The lubricant is preferably present in an amount of from about 0.01% to about 1 wt %, and most preferably about 0.23% w/w, based on the weight of the powder composition (free flowing granulate).

Suitable lubricants or glidants for use in the solid dosage forms described herein include, but are not limited to, stearic acid, calcium hydroxide, talc, corn starch, sodium stearyl fumarate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

### Additional Excipients

The oral solid dosage forms of the present invention may also contain pharmaceutically acceptable excipients such as fillers, diluents, lubricants, surfactants, glidants, binders, dispersing agents, suspending agents, disintegrants, viscosity-increasing agents, film-forming agents, granulation aid, flavoring agents, sweeteners, coating agents, solubilizing agents, and combinations thereof.

The pharmaceutical composition of the present invention can comprise the API and one or more pharmaceutically acceptable excipients in addition to the diluent and binder. Such excipients include but are not limited to a complexing agent, suspending agent, flavoring agent, sweetening agent, disintegrating agent, dispersing agent, surfactant, lubricant, colorant, solubilizer, moistening agent, plasticizer, stabilizer, wetting agent, anti-foaming agent, antioxidant, preservative, or one or more combination thereof.

Suitable preservatives include, for example, potassium sorbate, parabens (e.g., methylparaben and propylparaben) and their salts, benzoic acid and its salts, other esters of para hydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Suitable viscosity enhancing agents for the pharmaceutical compositions of the invention described herein include, but are not limited to, methyl cellulose, xanthan gum, carboxymethylcellulose, hydroxypropyl cellulose, hydroxypropylmethyl cellulose, Plasdone® S-630, carbomer, polyvinyl alcohol, alginates, acacia, chitosans and combinations thereof. The concentration of the viscosity-enhancing agent will depend upon the agent selected and the viscosity desired.

In certain embodiments and alkalizing agent or a buffer is included in the pharmaceutical composition, with suitable alkalizing agents and buffers including but not limited to NaOH, KOH, triethylamine, meglumine, L-Arginine, sodium phosphate buffer (either sodium phosphate tribasic, sodium phosphate dibasic, sodium phosphate monobasic, or o-phosphoric acid), sodium bicarbonate, and mixtures of any of the foregoing.

These additional ingredients may be incorporated extra granularly (after wet granulation), or may be wet granulated along with, e.g., the diclofenac, binder and diluent.

### Powder Sachet Processing

In a preferred embodiment, the powder sachets used in the invention are made by a wet granulation process in which the diclofenac powder is added to an alcoholic or hydroalcoholic solvent mixture, e.g., a solvent mixture of methanol and water, alone or with the binder. The solvent system of methanol and water may be prepared by using a methanol/water ratio of 80:20, 85:15, 90:10 or 95:05, v/v. In a particularly preferred embodiment, the solvent system of methanol and water is prepared by using a methanol:water ratio of about 90:10 v/v. The selection of solvent depends upon the solubility of diclofenac or diclofenac salt in the solvent. At the same time, the solvent system should keep mannitol undissolved. Also, the binder (e.g., PEG) has to dissolve during granulation. It has been found that several solvents in combination with water such as acetone, IPA, ethanol and DCM are useful, but the above-mentioned methanol/water solvent system worked best in terms of ease of granulation and impurity profile.

In certain preferred embodiments, a pharmaceutically acceptable binder (e.g., polyethylene glycol) is added in solvent system of methanol and water. The binder (e.g., polyethylene glycol) may be added in an amount from about 0.90% to about 1% of the total quantity of ingredients in the mixture (e.g., drug and binder, or drug/binder/diluent), to form a clear solution. The active pharmaceutical ingredient (API), e.g., diclofenac potassium, may be added in this mixture slowly by using constant mechanical stirring. The rate of addition of API to binder solution is evaluated, and stirring is continued until a white colored uniform dispersion free from lumps is obtained.

A preferred method of manufacturing the compositions of the present invention is wet granulation. For wet granulation, the binder can be added into a solution by using a solvent system of methanol and water. The solvent is usually methanol, water, or a mixture of both. The actual granulation is performed in either a high-shear, or low-shear type mixer. Low-shear granulation requires cheaper equipment and produces a more porous granule. High-shear granulation is faster and affords good control over particle size.

A rapid mixer granulator may be used for the wet granulation process in which the wet granulation is performed by using binder solution with added in dry diluent, e.g., mannitol (Mannogem® Granular). Preferably, mannitol may be used with coarse particle size, preferably having an average particle size of about 400 microns. Drug binder solution may be added into the rapid mixer granulator with flow rate of 180 gm/min. Additional solvent may be used to rinse the drug binder solution mixer vessel, and may be added in rapid mixer granulator. Wet granules are withdrawn and loaded on a fluidized bed dryer. The drying process may be carried out in the same vessel (a fluid bed granulator), wherein the wet mass is fluidized by dry air inside a chamber. The air flow is keep minimum so that drying process move smoothly. The total drying time required for completion of process is generally about 50-60 min. The process requires expensive equipment, but is simpler and produces a very porous low-density granule, which can result in faster drug dissolution. Slow drug dissolution is sometimes a problem associated with wet granulation, as the active ingredient is locked into the granule, and initial tablet disintegration liberates the granules rather than the primary drug particles. In contrast, in this embodiment, the diluent has a coarser particle size and after addition of the drug binder dispersion into the rapid mixer granulator, it produces a thin layer of drug on diluent i.e. diclofenac on mannitol (e.g., the resultant particles are agglomerated). Therefore, there is much less chance of drug entrapment during wet granulation.

In wet granulation, particle size enlargement is achieved by aggregating the powder particles under high pressure, then milling the material to the desired size. After completion of granulation, the resultant material is passed through various sieves. ASTM Sieve size 20, 30, 40, 60, 80, 120 & 140 may be used and the material which is retained on sieve 20 may be subjected to a further milling process.

In preferred embodiments, the drug, binder and diluent may be wet-granulated as described herein such that agglomerated particles are produced. In certain embodiments, a portion of the drug, binder and/or diluent may be added extra-granularly, i.e., without being included in the wet granulation process.

In preferred embodiments, the water content of the final product (free flowing powder granulate) is preferably less than about 2%.

### Formulations

In certain embodiments, the drugs may be formulated in a pharmaceutically acceptable oral dosage form. Oral dosage forms may include but are not limited to, oral solid dosage forms and oral liquid dosage forms. Oral solid dosage forms may include but are not limited to sachets, tablets, capsules, caplets, powders, pellets, multiparticulates, beads, spheres and/or any combinations thereof. These oral solid dosage forms are formulated as immediate release, but may further include controlled release, sustained (extended) release or modified release components.

### Final Powdered Sachet Product

The powder sachets used in the methods of this invention can be produced by various methods including wet granulation, drying, milling, blending and lubrication processes. A suitable product may be produced by wet granulation.

In one embodiment the powdered sachet is produced by wet granulation and is characterized by the presence of diclofenac particles having one of the particle size distributions described above. In another embodiment the product is characterized by the total amount of powder used to fill a sachet, which is preferably greater than 500, 600, 700 or 800 mg., and/or less than 1800, 1600, 1400, 1200, or 1000 mg, based on a 50 mg diclofenac sachet. A preferred amount of powder is about 900 mg and the amount preferably does not vary outside the 865-925 mg/sachet range per package.

In still another embodiment, the invention may be characterized by the solubility of the product in water, the amount of water required to solubilize the product, and the time required to solubilize the product in a given amount of water. Therefore, in one embodiment, a unit dose of the sachet is greater than 75% or 85% soluble or is completely soluble in 50 ml of water at 25 degrees Celsius. In another embodiment, the unit dose is greater than 75% or 85% solubilized or is completely solubilized in 50 ml of water with stirring at 25 degrees Celsius in less than 3 minutes. This optimized solubility seems to restrict absorption to a shorter part of the gastrointestinal tract, most likely contributing to the faster absorption rate and to the lower variability in the absorption compared to immediate release diclofenac potassium tablets.

This embodiment is a powder for solution product and therefore requires a water-soluble excipient. Microcrystalline cellulose (MCC) and other insoluble excipients are therefore ruled out. Mannitol also has negative heat of solution and gives cooling sensation required for taste masking.

Dissolution times for the powder for oral solution dosage forms of the present invention, when tested according to USP 28<711>, based on the time it takes to dissolve 90 or 95 wt % of the drug substance, are preferably less than about 5 minutes, 3 minutes, or even 2 minutes, and greater than about 1 or 2 minutes. In a preferred embodiment, the dissolution profile of the dosage forms of the present invention is as follows: not less than 85, 90 or 95% after 3 minutes in simulated intestinal fluid (i.e. water) at pH 6.8.

The final sachet product preferably also preferably includes a sweetener (a sugar or a non-sugar sweetener) and/or a flavorant in order to impart acceptable taste to the sachet product. The final sachet product is preferably free of sugar (saccharose). In certain preferred embodiments, the final product preferably includes as sweeteners one or more pharmaceutically acceptable non-sugar sweeteners known to those skilled in the art such as Sucralose®, and preferably includes one or more flavoring agents. The flavoring agents may be compounds such as anise and/or peppermint.

Practically any container that maintains hermetic conditions could be used for packaging the powder sachets, though preferably the container consists of a sachet that is hermetically sealed in four directions to maintain the product in hermetic conditions during storage. The sachet is preferably made from a four-layer coupled Opaque Laminating Base Paper (OLB) paper/PE/Aluminium foil/PE having a width of 295 mm in which the weight of the paper is from about 0.475 to about 0.525 g/100 cm², the weight of the aluminum is from about 0.203 to about 0.229 g/100 cm², and the weight of the polyethylene is from about 0.295 to about 0.365 g/100 cm².

### Other Ingredients

In further embodiments, a powder comprising the active agent(s) of the present invention formulations described herein may be formulated to comprise one or more pharmaceutical excipients and flavors. Such a powder may be prepared, for example, by mixing the active agent(s) of the present invention formulation and optional pharmaceutical excipients to form a bulk blend composition. Additional embodiments also comprise a suspending agent and/or a wetting agent. This bulk blend is uniformly subdivided into unit dosage packaging or multi-dosage packaging units. The term “uniform” means the homogeneity of the bulk blend is substantially maintained during the packaging process.

### Tablets and Capsules

In some embodiments, the solid dosage forms of the present invention may be in the form of a tablet, (including a suspension tablet, a bite-disintegration tablet, a rapid-disintegration tablet, an effervescent tablet, or a caplet), a pill, a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. Additionally, pharmaceutical formulations of the present invention may be administered as a single tablet or capsule or in multiple tablet or capsule dosage forms. In some embodiments, the pharmaceutical formulation is administered in two, or three, or four sachets, capsules or tablets.

In certain embodiments, oral solid dosage forms of the free flowing powder granulate of the present invention compositions, as described herein, include one or more pharmaceutically acceptable additives such as a compatible carrier, binder, complexing agent, ionic dispersion modulator, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In still other aspects, using standard coating procedures, such as those described in Remington's Pharmaceutical Sciences, 20th Edition (2000), a film coating is provided around the portion of the API to be included in such oral solid dosage form.

Suitable carriers for use in such oral solid dosage forms described herein include, but are not limited to acacia, gelatin, colloidal silicon dioxide, calcium glycerophosphate, calcium lactate, maltodextrin, glycerin, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose (e.g., Avicel®, Avicel® PH101, Avicel® PH102, Avicel® PH105, etc.), cellulose powder, dextrose, dextrates, dextrose, dextran, starches, pregelatinized starch, hydroxypropylmethylcellulose (HPMC), hydroxypropylmethylcellulose phthalate, hydroxypropylmethylcellulose acetate stearate (HPMCAS), sucrose, xylitol, lactitol, mannitol, sorbitol, sodium chloride, polyethylene glycol, and the like.

If needed, suitable disintegrants for use in such oral solid dosage forms described herein include, but are not limited to, natural starch such as corn starch or potato starch, a pregelatinized starch such as National 1551 or Amijel®, or a sodium starch glycolate such as Promogel® or Explotab®, a cellulose such as a wood product, microcrystalline cellulose, e.g., Avicel®, Avicel® PH101, Avicel® PH102, Avicel® PH105, Elcema® P100, Emcocel®, Vivacel®, Ming Tia®, and Solka-Floc®, Ac-Di-Sol, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crosspovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

Binders impart cohesiveness to solid oral dosage form formulations: for powder-filled capsule formulation, they aid in plug formation that can be filled into soft or hard shell capsules and in tablet formulation, binders ensure that the tablet remains intact after compression and help assure blend uniformity prior to a compression or fill step. Materials suitable for use as binders in the solid dosage forms described herein include, but are not limited to, carboxymethylcellulose, methylcellulose (e.g., Methocel®), hydroxypropylmethylcellulose (e.g. Hypromellose USP Pharmacoat-603, hydroxypropylmethylcellulose acetate stearate (Aqoate HS-LF and HS), hydroxyethylcellulose, hydroxypropylcellulose (e.g., Klucel®), ethylcellulose (e.g., Ethocel®), and microcrystalline cellulose (e.g., Avicel®), microcrystalline dextrose, amylose, magnesium aluminum silicate, polysaccharide acids, bentonites, gelatin, polyvinylpyrrolidone/vinyl acetate copolymer, crosspovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinylpyrrolidone (e.g., Povidone® CL, Kollidon® CL, Polyplasdone® XL-10, and Povidone® K-12), larch arabogalactan, Veegum®, polyethylene glycol, waxes, sodium alginate, and the like. In general, binder levels of 20-70% are used in powder-filled gelatin capsule formulations. Binder usage level in tablet formulations is a function of whether direct compression, wet granulation, roller compaction, or usage of other excipients such as fillers which itself can act as moderate binders are used. Formulators skilled in the art can determine the binder level for the formulations, but binder usage level of up to 70% in tablet formulations is common.

Suitable lubricants or glidants for use in the solid dosage forms described herein include, but are not limited to, stearic acid, calcium hydroxide, talc, corn starch, sodium stearyl fumarate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

Non-water-soluble diluents are compounds typically used in the formulation of pharmaceuticals, such as calcium phosphate, calcium sulfate, starches, modified starches and microcrystalline cellulose, and micro cellulose (e.g., having a density of about 0.45 g/cm³, e.g. Avicel, powdered cellulose), and talc.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat) 10®), sodium oleate, sodium lauryl sulfate, magnesium stearate, sodium docusate, triacetin, vitamin E TPGS, sodium lauryl sulfate, sorbitan monooleate, polyoxyethylene sorbitan monooleate, polysorbates, poloxamers, bile salts, glyceryl monostearate, copolymers of ethylene oxide and propylene oxide, e.g., Pluronic® (BASF), and the like.

Suitable suspending agents for use in such solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 18000, vinylpyrrolidone/vinyl acetate copolymer (S630), sodium alginate, gums, such as, e.g., gum tragacanth and gum acacia, guar gum, xanthans, including xanthan gum, sugars, cellulosic, such as, e.g., sodium carboxymethylcellulose, methylcellulose, sodium carboxymethylcellulose, hydroxypropylmethylcellulose, hydroxyethylcellulose, polysorbate-80, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), butyl hydroxyanisole (BHA), sodium ascorbate, Vitamin E TPGS, ascorbic acid, sorbic acid and tocopherol.

Immediate-release tablet formulations may be prepared by combining the free-flowing powder granulate with a disintegrant or super disintegrant such as croscarmellose sodium and different grades of microcrystalline cellulose.

### Liquid Dosage Forms

In other embodiments of the invention, the free flowing powder granulate is divided into unit doses which are dissolved into a liquid dosage form, e.g., prior to oral administration. Such liquid dosage forms may be in the form of pharmaceutically acceptable emulsions, syrups, elixirs, suspensions, and solutions, which may contain an inactive diluent, such as water. Pharmaceutical compositions may be prepared as liquid suspensions or solutions using a sterile liquid, such as but not limited to water, an al cohol, and combinations of these pharmaceutically together with optional suitable surfactants, suspending agents and emulsifying agents. Such liquid dosage forms may include liquid carriers such as water, alcohols (such as, but not limited to, ethanol, isopropyl alcohol, hexadecyl alcohol, glycerol, and propylene glycol), ethers, such as, but not limited to, poly(ethylene glycol). In addition to the active agent(s) of the present invention particles, the liquid dosage forms may comprise additives, such as (a) disintegrating agents; (b) dispersing agents; (c) wetting agents; (d) at least one preservative, (e) viscosity enhancing agents, (f) at least one sweetening agent, and (g) at least one flavoring agent.

Examples of disintegrating agents for use in the aqueous suspensions and dispersions include, but are not limited to, a starch, e.g., a natural starch such as corn starch or potato starch, a pregelatinized starch such as National 1551 or Amijel®, or sodium starch glycolate such as Promogel® or Explotab®; a cellulose such as a wood product, microcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crosspovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein are known in the art and include, for example, hydrophilic polymers, electrolytes, Tween® 60 or 80, PEG, polyvinylpyrrolidone (PVP; commercially known as Plasdone®), and the carbohydrate-based dispersing agents such as, for example, hydroxypropylcellulose and hydroxypropylcellulose ethers (e.g., HPC, HPC-SL, and HPC-L), hydroxypropylmethylcellulose and hydroxypropylmethylcellulose ethers (e.g. HPMC K100, HPMC K4M, HPMC K15M, and HPMC K100M), carboxymethylcellulose sodium, methylcellulose, hydroxyethylcellulose, hydroxypropylmethylcellulose phthalate, hydroxypropylmethylcellulose acetate stearate, noncrystalline cellulose, magnesium aluminum silicate, triethanolamine, polyvinyl alcohol (PVA), polyvinylpyrrolidone/vinyl acetate copolymer (Plasdone®, e.g., S-630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908°, also known as Poloxamine 908°, which is a tetrafunctional block copolymer derived from sequential addition of propylene oxide and ethylene oxide to ethylenediamine (BASF Corporation, Parsippany, N.J.)). In other embodiments, the dispersing agent is selected from a group not comprising one of the following agents: hydrophilic polymers; electrolytes; Tween® 60 or 80; PEG; polyvinylpyrrolidone (PVP); hydroxypropyl cellulose and hydroxypropyl cellulose ethers (e.g., HPC, HPC-SL, and HPC-L); hydroxypropyl methylcellulose and hydroxypropyl methylcellulose ethers (e.g. HPMC K100, HPMC K4M, HPMC K15M, HPMC K100M, and Pharmacoat® USP 2910 (Shin-Etsu)); carboxymethylcellulose sodium; methylcellulose; hydroxyethylcellulose; hydroxypropylmethylcellulose phthalate; hydroxypropylmethylcellulose acetate stearate; non-crystalline cellulose; magnesium aluminum silicate; triethanolamine; polyvinyl alcohol (PVA); 4-(1,1,3,3-tetramethyl butyl)-phenol polymer with ethylene oxide and formaldehyde; poloxamers (e.g., Pluronics F68®, F88®, and F108®, which are block copolymers of ethylene oxide and propylene oxide); or poloxamines (e.g., Tetronic 908®, also known as Poloxamine 908®).

Wetting agents (including surfactants) suitable for the aqueous suspensions and dispersions described herein are known in the art and include, but are not limited to, acetyl alcohol, glycerol monostearate, polyoxyethylene sorbitan fatty acid esters (e.g., the commercially available Tweens® such as e.g., Tween 20® and Tween 80® (ICI Specialty Chemicals)), and polyethylene glycols (e.g., Carbowaxs 3350® and 1450®, and Carpool 934® (Union Carbide)), oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphatidylcholine and the like.

Suitable preservatives for the aqueous suspensions or dispersions described herein include, for example, potassium sorbate, parabens (e.g., methylparaben and propylparaben) and their salts, benzoic acid and its salts, other esters of para hydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

### Release of the API

In some embodiments, the release of the API of the present invention free-flowing particles can be modified with a modified release coating, such as an enteric coating using cellulose acetate phthalate or a sustained release coating comprising copolymers of methacrylic acid and methylmethacrylate. In one embodiment, the enteric coating may be present in an amount of about 0.5 to about 15 wt %, more specifically, about 8 to about 12 wt %, based on the weight of, e.g., the spray layered particles. In one embodiment, the spray layered particles coated with the delayed and/or sustained release coatings can be filled in a modified release capsule in which both enteric-coated and immediate release the active agent(s) of the present invention beads are filled into a soft gelatin capsule. Additional suitable excipients may also be filled with the coated particles in the capsule. The uncoated particles release the active agent of the present invention immediately upon administration while the coated particles do not release the active agent(s) of the present invention until these particles reach the intestine. By controlling the ratios of the coated and uncoated particles, desirable pulsatile release profiles may be obtained. In some embodiments, the ratios between the uncoated and the coated particles are e.g., 20/80, or 30/70, or 40/60, or 50/50, w/w to obtain desirable release.

In certain embodiments, a pulsatile release the API of the present invention formulation comprises a first portion of the dosage unit comprising the free-flowing powder granulate or a product made therefrom (e.g., sachets, tablets, capsules, etc.) and a second portion of the dosage unit comprises the API in modified, delayed, sustained or controlled release form. The second portion of the dosage unit may be prepared from API granules made from a spray drying or spray granulated procedure or a formulation made from the API comprising the API spray layered onto a substrate (e.g., a bead) or the API incorporated into an immediate release granulate which is then coated with delayed, enteric or sustained-release coatings known to those skilled in the art. Such formulations would still employ the benefits of the rapidly bioavailable (first) portion of the unit dose comprising the pharmaceutical composition of the invention, along with the benefits of longer activity afforded by the second portion of the dosage unit.

### Detailed Description of Preferred Embodiments

The following examples are put forth so as to provide those of ordinary skill in the art with a complete disclosure and description of how the compounds claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at room temperature, and pressure is at or near atmospheric.

### Example 1: Representative 900 mg—Powder Sachet Formulation

Table 2 describes the composition of a representative 900 mg powder sachet formulation containing 50 g of diclofenac potassium that is suitable for practicing the present invention.

TABLE 2

| | | Quantity/Packet | |
|---|---|---|---|
| Sr. No. | Name of the ingredient | (in mg) | in % w/w |
| 1. | Mannitol (Mannogem ® | 721.00 | 81.38 |
| | Granular) | | |
| 2. | Diclofenac Potassium | 50.00 | 5.64 |
| 3. | Polyethylene Glycol | 8.00 | 0.90 |
| | 8000 (Carbowax Sentry | | |
| | PEG 8000) | | |
| 4. | Methanol | qs | qs |
| 5. | Purified Water | qs | qs |
| 6. | Star Anise Flavour SD | 15.00 | 1.69 |
| 7. | Sucralose | 55.00 | 6.21 |
| 8. | Peppermint Flavour | 35.00 | 3.95 |
| 9. | Glyceryl Dibehenate | 2.00 | 0.23 |

| | | |
|---|---|---|
| Final Weight | 886.00 | 100.00 |

The FIGURE is a flow diagram illustrating a wet granulation method and sequence of mixing employed in making 900 mg powder sachets of the invention that contain 50 mg of diclofenac potassium.

### Example 2: Manufacturing Process for 900 mg Powder Sachets Containing 50 mg of Diclofenac Potassium

A representative process for manufacturing 900 mg powder sachets containing 50 mg of diclofenac potassium is set forth below, using the equipment set forth in Table 3 below. The manufacture is performed under controlled temperature and relative humidity according to the following process.

TABLE 3
Step Description

| 1 | Dispensing: Dispense all the ingredients as per formula sheet. | | | |
|---|---|---|---|---|
| 2 | Drug-Binder Dispersion Preparation: | | | |
| | 2.1 Solvent System Preparation: Dispense 2.508 kg of | | | |
| | Methanol and 0.358 kg of Purified water and mix together | | | |
| | to obtain uniform solvent system. Additionally, dispense | | | |
| | 0.230 kg of Methanol (i.e. 0.55% w/w of Mannogem ^{®} | | | |
| | Granular) for rinsing purpose. | | | |
| | 2.2 Drug-Binder Dispersion Preparation: | | | |
| | Add 0.464 kg of Polyethylene Glycol 8000 to the solvent | | | |
| | system of step 2.1 and stir to obtain clear solution. | | | |
| | Then add 2.901 kg of Diclofenac Potassium to the | | | |
| | obtained clear solution under constant mechanical stirring. | | | |
| | Continue stirring until white colored uniform dispersion | | | |
| | free from lumps is obtained. Use additionally dispensed | | | |
| | Methanol for rinsing purpose. | | | |
| | Description of binder solution: White colored | | | |
| | uniform dispersion free from lumps. | | | |
| 3 | Sifting: Sift 41.828 kg of Mannitol through #20 | | | |
| | ASTM and collect it in a polybag. | | | |
| 4. | Load the material of step 3 in Rapid Mixer Granulator. | | | |
| | Granulate the dry mix with Drug-Binder dispersion | | | |
| | of step 2 through peristaltic pump with controlled rate. | | | |
| | Set parameters for granulation as mentioned below. | | | |
| | Drug-Binder dispersion addition rate approximately | | | |
| | 150-200 g/min | | | |
| | Granulation Parameters: | | | |
| | RMG Parameters | | Set Speed | |
| | | Time | Impeller | Chopper |
| | Stage | (sec.) | (RPM) | (RPM) |
| | Dry Mix | 60 | To be | Off |
| | | | recorded | |
| | Drug-Binder | To be | To be | Off |
| | Dispersion | recorded | recorded | |
| | addition* | | | |
| | Wet Mixing | 40 | To be | Off |
| | | | recorded | |

| | *Additionally dispensed 0.230 kg of Methanol (i.e. 0.55% | |
|---|---|---|
| | w/w of Mannogem ^{®} Granular) shall be used for rinsing | |
| | of the drug-binder dispersion vessel. This at a controlled | |
| | rate using a rinsed solution shall be added through a silicon | |
| | tube peristaltic pump by keeping the same machine | |
| | parameters as that of the drug-binder solution addition stage. | |
| 5 | Discharge the wet granules from RMG Bowl and subject to | |
| | drying by loading them into the Fluidized Bed Dryer. LOD is | |
| | monitored and which is not more than 0.6% | |
| | (at 105° C. for 5 minutes) | |
| | LOD: NMT 0.6 % (at 105° C. for 5 minutes) | |
| | B.D of blend: 0.6666 g/mL | |
| | T.D of blend: 0.7692 g/mL | |
| | PSD of granules: | |
| | #ASTM Sieve | % Cumulative Retained |
| | 20 | 0.57 |
| | 30 | 35.93 |
| | 40 | 62.12 |
| | 60 | 86.73 |
| | 80 | 95.93 |
| | 100 | 98.28 |
| | 140 | 99.70 |
| | Collector | 100.00 |
| | Description: White to off-white, free flowing, granular powder. | |
| 6 | Milling: Sift the dried granules through #20 ASTM. Mill the | |
| | #20 ASTM retained granules through multi mill fitted with | |
| | 1.0 mm screen at appropriate speed (Knife forward direction). | |
| | Sift the milled granules through #20 ASTM. On further | |
| | retention of granules, mill through multi mill fitted with 0.5 | |
| | mm screen at appropriate speed (Knife forward direction) and | |
| | sift the milled granules through #20 ASTM. Collect all portions | |
| | of #20 ASTM passed granules into double lined polybag. | |
| | Note: | |
| | If further retention observes, mill the retained granules through | |
| | Multi mill fitted with 0.5 mm screen at appropriate speed | |
| | (Knife forward direction). Sift the milled granules | |
| | through #20 ASTM and mix with #20 ASTM passed granules | |
| | of initial step. Continue the step till #20 ASTM retains | |
| | become negligible (Negligible quantity shall be less than or | |
| | equal to 0.1% w/w of the theoretical blend). | |
| 7. | Calculate the extra granular materials (Sucralose, | |
| | Peppermint Flavor, Star Anise Flavor and Glyceryl | |
| | Dibehenate) quantity based on the yield of dried granules. | |
| 8 | Sifting: | |
| | 8.1. Sift the dried granules of step 6 through #60 | |
| | ASTM and collect the #60 ASTM passed and | |
| | retained portion in separate polybags. | |
| | 8.2. Co-sift 3.191 kg of Sucralose, 2.030 kg | |
| | of Peppermint Flavor and 0.870 kg of Star Anise Flavor | |
| | SD through #40 ASTM Sieve and collect in polybag. | |
| | 8.3. Co-sift the #60 ASTM passed portion of | |
| | granules (of step 8.1) and sifted materials of | |
| | step 8.2 through #40 ASTM Sieve two times. | |
| 9 | Blending: | |
| | Load the materials of step 8 for blending | |
| | purpose in Conta blender with below sequence; | |
| | 9.1. First, load approximately 50% quantity of #60 ASTM | |
| | retained granules of step 8.1 into the blender. | |
| | 9.2. Then add co-sifted material of step 8.3. | |
| | 9.3. Finally, add remaining approximately 50% quantity | |
| | of #60 ASTM retained granules of step 8.1 into the blender. | |
| | 9.4. Perform blending for 24 min at 15 RPM. | |
| 10 | Lubrication: | |
| | Sift 0.116 kg of Glyceryl Dibehenate through #60 ASTM and | |
| | add it with the above material of step 9. Carry out the | |
| | lubrication for 8 minutes at 15 RPM in Conta blender. | |
| | B.D of blend: 0.7272 g/mL | |
| | T.D of blend: 0.8333 g/mL | |
| | Occupancy in 5 L blender at R&D Stage: ~69% | |
| | PSD of granules: | |
| | #ASTM Sieve | % Cumulative Retained |
| | 20 | 0.18 |
| | 30 | 29.88 |
| | 40 | 53.13 |
| | 60 | 75.03 |
| | 80 | 83.01 |
| | 100 | 85.76 |
| | 140 | 89.96 |
| | Collector | 100.00 |

| | |
|---|---|
| | Description: White to off-white, free flowing, granular powder. |
| 10. | Sachet Filling: Fill the blend of step 9 in packets having |
| | dimension of 81 mm × 44 mm. The packet should be sealed |
| | properly from all sides. |

### Example 3: Evaluation of Different Grade of Diluent Mannitol

In this study, another grade of Mannitol was evaluated. The average particle size of Pearlitol 400 DC is 360μ. Another grade of Mannitol i.e., Mannogem® Granular (Source: SPI Pharma) with average particle size of −400 microns was chosen. The manufacturing process executed was same as described for the previous batch i.e., wet granulation approach with drug and binder solution mixture.

The composition of the formulation, brief manufacturing process and comparative analysis is set forth in Table 4 below.

TABLE 4

| Unit Composition | | 50 mg |
|---|---|---|
| Batch Nos. | | IQ014(010)19 & IQ014(010)21 |
| Sr. No. | Ingredients | Mg/Sachet |

Intragranular

| | | |
|---|---|---|
| 1 | Mannitol | 721.00 |
| | (Mannogem ^{®} | |
| | Granular) | |
| 2 | Sucralose | 55.00 |
| | (Sucrex) | |

Binder Solution

| | | |
|---|---|---|
| 3 | Diclofenac | 50.00 |
| | Potassium* | |
| 4 | Polyethylene | 8.00 |
| | Glycol 8000 | |
| | (Carbowax | |
| | Sentry | |
| | PEG 8000) | |
| 5 | Methanol | QS |
| 6 | Purified | QS |
| | Water | |

Extragranular

| | | |
|---|---|---|
| 7 | Star Anise | 15.00 |
| | Flavour SD | |
| | (0499085)** | |
| 8 | Peppermint | 35.00 |
| | Flavour | |
| | (PL-0132) | |
| 9 | Glyceryl | 2.00 |
| | Dibehenate | |
| | Total Weight | 886.00 |

*Actual quantity of API taken based on Assay and LOD value
calculation and compensated with Mannitol
** Batch no. IQ014(010)19 does not contain Star Anise Flavour
SD (0499085).
Brief manufacturing Process
1. Diclofenac Potassium was dissolved in solvent mixture of ,ethanol
and purified water (Solvent in 9:1 ratio)-Solution A
2. Wet granulation was performed with Solution A as binder
solution with gradual addition to the mixture of Mannitol and
Sucralose (Care was taken to avoid loss of solution A while
addition to the dry mix).
3. Granules of step 2 were dried, milled and mixed with
extragranular materials and processed further. During the drying
stage, the fluidization of material was kept to a minimum
to avoid process loss through filter bag.
Comparative Analytical Results:

| | | Test | | |
|---|---|---|---|---|
| | | Formulation | | |
| | | (Formulation | Test Formulation | |
| | | with Pearlitol | (Formulation | |
| | | 400 DC, Wet | with Mannogem ^{®} | |
| | | granulation | Granular, Wet | |
| | CAMBIA ^{®}: | with drug | granulation | |
| | Reference | containing | with drug | |
| | Product- | binder | containing | |
| Test | 50 mg | solution) | binder solution) | |
| Batch | Lot S004 | IQ014 | IQ014 | IQ014 |
| No. | | (010)17 | (010)19 | (010)21 |
| De- | White, free | White, free | White, free | White, free |
| scription | flowing, | flowing, | flowing, | flowing, |
| | granular | granular | granular | granular |
| | powder. | powder. | powder. | powder. |
| Assay | 100.0 | 98.4 | 100.8 | 98.7 |
| (%) | | | | |
| CU(%) | — | 98.2 | 100.3 | 98.3 |
| | | (AV 2.01) | (AV 2.16) | (AV 2.86) |
| Disso- | Media: 400 mL, pH 6.8, 0.05M | | | |
| lution | phosphate buffer, USP Type II, 75 RPM | | | |
| Time | | | | |
| (min) | % Drug Release | | | |
| 2.5 | 99(97-103) | 91(88-94) | 84(80-87) | 89(85-93) |
| 5 | 100(97-103) | 98(97-99) | 95(92-97) | 98(95-102) |
| 7.5 | 100(98-103) | 99(98-99) | 98(97-99) | 100(99-102) |
| 10 | 101(98-103) | 99(98-99) | 99(97-100) | 99(98-102) |

The particle size distribution data of the formulation is set forth in Table 5 below.

TABLE 5
Particle Size Distribution Data

| | % Cumulative Retained | |
|---|---|---|
| | Plain Diluent | Wet Granulation |
| | Mannogem ^{®} | with Mannogem ^{®} |
| ASTM Sieve (#) | Granular | IQ014(010)21 |
| 20 (850 μ) | 0.30 | 2.31 |
| 30 (600 μ) | 18.28 | 25.00 |
| 40 (425 μ) | 14.04 | 48.05 |
| 60 (250 μ) | 73.64 | 67.11 |
| 80 (180 μ) | 89.22 | 74.17 |
| 100 (150 μ) | 95.72 | 82.94 |
| 140 (106 μ) | 98.66 | 93.38 |
| 200 (75 μ) | 99.28 | 99.97 |
| Collector (<75 μ) | 100.00 | 100.00 |
| D90 | 720 | 760 |
| D50 | 380 | 410 |
| D10 | 180 | 120 |

There was no impact on CQAs like Assay and CU of the formulation with the use of another grade of mannitol (Mannogem® Granular) when compared to Pearlitol 400DC. The Granular were also comparable with that of reference formulation. In addition, the average particle size of Mannogem® Granular is −400 microns as compared to 360 microns of Pearlitol 400DC, which is well outside the average particle size of the RLD.

### Example 4: Extragranular Component in the Formulation

In Example 4, the formulation contains sucralose as an extragranular component of the formulation. The composition of the formulation is set forth in Table 6 below.

| Unit Composition | | 50 mg |
|---|---|---|
| Sr. No. | Name of the ingredient | Quantity/Packet (mg) |
| Intragranular Ingredients | | |
| 1. | Mannitol | 721.00 |
| Binder Solution | | |
| 2. | Diclofenac Potassium* | 50.00 |
| 3. | Polyethylene Glycol 8000 | 8.00 |
| 4. | Methanol | Qs |
| 5. | Purified Water | |
| Extra granular Ingredients | | Qs |
| 6. | Star Anise Flavour SD | 15.00 |
| 7. | Sucralose | 55.00 |
| 8. | Peppermint Flavour | 35.00 |
| 9. | Glyceryl Dibehenate | 2.00 |

| | |
|---|---|
| Weight of Unit Dosage | 886.00 |

The manufacturing process was as follows:

1. Diclofenac Potassium was dissolved in solvent mixture of methanol and purified water (Solvent in 8.75:1.25 ratio)—Solution A
2. Wet granulation was performed with solution A as the binder solution with gradual addition to the Mannitol (Care was taken to avoid loss of solution A while addition to the dry mix).
3. Granules of step 2 were dried to achieve LOD of NMT 1.0% w/w. The dried granules were milled sifted to get a uniform particle size.
4. The sifted granules of step 3 were blended with extragranular materials Sucralose, Star Anise Flavour SD and Peppermint Flavour in a blender.
5. The blend of step 4 was lubricated with Glyceryl Dibehenate and filled in a sachet.

The dissolution of the formulation as compared to a reference product is set forth in Table 7 below:

TABLE 7

| | CAMBIA ^{®}: Reference | |
|---|---|---|
| Test | Product-50 mg | Test Formulation |
| Dissolution | Media: 400 mL, pH 6.8, 0.05M phosphate | |
| | buffer, USP Type II, 75 RPM | |
| Time (min) | % Drug Release | |
| 2.5 | 99(97-103) | 100(91-109) |
| 5 | 100(97-103) | 106(105-109) |
| 7.5 | 100(98-103) | 106(104-106) |
| 10 | 101(98-103) | 105(104-107) |

### CONCLUSION

All of the percentages in the Specification, and specifically in the Examples provided above, are expressed as w/w unless otherwise indicated.

In the preceding Specification, the invention has been described with reference to specific exemplary embodiments and examples thereof. It will, however, be evident that various modifications and changes may be made thereto without departing from the broader spirit and scope of the invention as set forth in the claims that follow. The Specification and drawings are accordingly to be regarded in an illustrative manner rather than a restrictive sense.

What is claimed is:

1. A pharmaceutical composition for oral administration comprising a free-flowing granular powder comprising agglomerated particles of diclofenac or a pharmaceutically acceptable salt thereof; a pharmaceutically acceptable substantially non-hygroscopic, water soluble diluent consisting of mannitol having an average particle diameter above about 350 microns; an effective amount of a pharmaceutically acceptable binder to bind the diclofenac to the diluent; and one or more optional pharmaceutically acceptable excipients, wherein not less than 85% of the free-flowing granulate powder dissolves after 3 minutes in simulated intestinal fluid at pH 6.8.

2. The pharmaceutical composition of claim 1, which comprises from about 25 to about 75 mg of diclofenac or a pharmaceutically acceptable salt thereof, and the diluent is mannitol, the pharmaceutical composition comprising from about 300 to about 800 mg of mannitol having an average particle diameter above about 350 microns and the pharmaceutical composition has an average particle size from about 350 microns to about 900 microns.

3. The pharmaceutical composition of claim 1, wherein the free-flowing, granular powder comprises agglomerated particles of diclofenac and the diluent is prepared via wet-granulation.

4. The pharmaceutical composition of claim 1, wherein the composition is devoid of any alkalizer, buffer or base.

5. The pharmaceutical composition of claim 1, wherein the free-flowing, granular powder comprises agglomerated particles of the diclofenac, diluent and binder.

6. The pharmaceutical composition of claim 5, wherein the diluent comprises mannitol having an average particle diameter from about 400 to about 750 microns.

7. The pharmaceutical composition of claim 6, wherein the binder is polyethylene glycol.

8. The pharmaceutical composition of claim 5, wherein the free-flowing granular powder includes from about 5% to about 10% diclofenac, from about 60% to about 95% mannitol, and from about 0.1% to about 3% binder, w/w.

9. The pharmaceutical composition of claim 9, wherein the binder is polyethylene glycol.

10. The pharmaceutical composition of claim 5, further comprising an extragranular excipient selected from the group consisting of a lubricant, a flavorant, a sweetener, and mixtures of any of the foregoing.

11. The pharmaceutical composition of claim 1, wherein the free-flowing granular powder is contained in a sachet containing a therapeutically effective dose of diclofenac or a pharmaceutically acceptable salt thereof.

12. The pharmaceutical composition of claim 10, wherein the free-flowing granular powder is contained in a sachet containing a therapeutically effective dose of diclofenac or a pharmaceutically acceptable salt thereof.

13. The pharmaceutical composition of claim 1, wherein the free-flowing powder granulate is incorporated into a tablet or a capsule.

14. The pharmaceutical composition of claim 1, wherein the free-flowing powder granulate is incorporated into an oral solution, oral solution or suspension.

15. A method of treating acute pain in a mammal in need thereof, comprising orally administering the pharmaceutical composition of claim 1 to the mammal.

16. A method of treating a migraine with or without aura in a mammal in need thereof, comprising orally administering the pharmaceutical composition of claim 12 to the mammal.

17. The pharmaceutical composition of claim 10, which has a water content of less than about 2%.

18. The pharmaceutical composition of claim 12, wherein from about 500 mg to about 1800 mg of the pharmaceutical composition is contained in the sachet.

19. The pharmaceutical composition of claim 19, wherein the sachet contains from about 865 mg to about 925 mg of the free-flowing powder granulate containing about 50 mg diclofenac.

20. A method of preparing an oral dosage form of diclofenac, comprising adding a pharmaceutically acceptable binder to an alcoholic or hydroalcoholic solvent; adding diclofenac or a pharmaceutically acceptable salt thereof to the binder/solvent mixture to obtain a drug-binder dispersion; granulating a substantially non-hygroscopic water soluble diluent with the drug-binder dispersion; and drying the resultant mixture to obtain a free-flowing granular powder.

21. The method of claim 21, further comprising dividing the free-flowing granular powder into unit doses containing a dose of diclofenac from about 25 mg to about 75 mg, and filling the blend into suitably sized sachets such that each sachet contains from about 500 mg to about 1800 mg of the pharmaceutical composition.

22. The method of claim 22, further comprising adding a pharmaceutically acceptable lubricant to the free-flowing granular powder prior to filling the blend into the sachets.

23. The method of claim 23, further comprising adding one or more pharmaceutically acceptable excipients extra-granularly to the free-flowing granular powder.
